(19) United States
(12) Patent Application Publication
(10) Pub. No.: US 2022/0227858 A1
(43) Pub. Date: Jul. 21, 2022

(54) CONTROLLING OF IMMUNE ACTIVATION BY SOLUBLE CLEVER-1

(71) Applicant: FARON PHARMACEUTICALS OY, Turku (FI)

(72) Inventors: Juho JALKANEN, Mietoinen (FI); Maija-Leena HOLLMÉN, Piispanristi (FI); Carlos Rogerio ("Roger") DE FIGUEIREDO, Helsinki (FI)

(73) Assignee: FARON PHARMACEUTICALS OY, Turku (FI)

(21) Appl. No.: 17/577,132

(22) Filed: Jan. 17, 2022

(30) Foreign Application Priority Data
Jan. 18, 2021 (FI) 20215058

(51) Int. Cl. C07K 16/28; A61P 37/06

(52) U.S. Cl. CPC C07K 16/28; A61K 2039/505; A61P 37/06

(57) ABSTRACT

Altering levels of soluble CLEVER-1 in circulation achieves either immune activation or immune-suppression of T cells in an individual. Soluble CLEVER-1 has been found to bind to activated T cells using Receptor of Activated Protein C Kinase 1 (RACK1) as its ligand.

### FIELD OF THE INVENTION

The present invention relates to altering levels of soluble CLEVER-1 in order to either achieve immune activation or immunosuppression of T cells in an individual, and also to affecting binding of soluble CLEVER-1 on lymphocytes. The present invention further relates to a method of affecting soluble CLEVER-1 for use in treatment of various diseases and disorders.

### BACKGROUND OF THE INVENTION

The human CLEVER-1 (Common Lymphatic Endothelial and Vascular Endothelial Receptor-1) is disclosed in the publication WO 03/057130. CLEVER-1 is also known as Stabilin-1 or Feel-1. CLEVER-1 is a large glycoprotein receptor that is expressed on the surface of lymphatic endothelial cells, sinusoidal endothelial cells and also alternatively activated immunosuppressive macrophages, often referred to as tumour associated macrophages, and which is involved in scavenging, angiogenesis and cell adhesion. It is also previously presented in the publication WO2010/122217 that blocking of CLEVER-1 on macrophages by CLEVER-1 specific antibodies reduce the size of malignant tumour and/or malignant tumour growth. It is also previously presented e.g. in the publication WO2017/182706 that blocking of CLEVER-1 on macrophages turns alternatively activated macrophages from an immunosuppressive (M2) phenotype to a pro-inflammatory (M1) phenotype.

The publication WO 03/057130 also discloses that CLEVER-1 mediates binding of other types of leukocytes such as monocytes and granulocytes to HEV-like vessels. Thus, by blocking the interaction of CLEVER-1 on vessels it became possible to control metastasis by preventing malignant cells that bind to CLEVER-1 from being taken up by the lymphatic vessels, and thus to prevent spread of the malignancy into the lymph nodes.

Prior art discloses methods to inhibit the action of CLEVER-1 on monocytes, macrophages and endothelial cells.

### SUMMARY OF THE INVENTION

It has now been found that CLEVER-1 is also present in large quantities in soluble form in the serum and lymph of end-stage cancer patients, and that this previously poorly known soluble form of the CLEVER-1 protein, thought to be inactive, is in fact a highly active and immunosuppressive protein in circulation. The role of soluble CLEVER-1 has not been known until now and altering levels of soluble CLEVER-1 as mean to either activate or inactivate T cells in an individual is an entirely new concept.

It is an object of the present invention to provide a method for controlling immune activation in an individual and hence a method of controlling and/or affecting levels of soluble CLEVER-1 in circulation and/or lymph for use in treatment of various diseases and disorders.

In circulation soluble CLEVER-1 (sCLEVER-1) can be found in exosomes, micro vesicles, free form, and bound to apoptotic bodies and cell debris. It has been now found that in all of these forms circulating soluble CLEVER-1 is an active immunosuppressive protein, not a macrophage or endothelial cell receptor as commonly thought. It has also been found that soluble CLEVER-1 binds to activated T cells using Receptor of Activated Protein C Kinase 1 (RACK1) as its ligand. RACK 1 is in all cells, but usually inactive within the cell. In activated T cells RACK1 is brought to the surface and soluble CLEVER-1 has now found to bind to it. Binding of soluble CLEVER-1 to RACK1 leads to cytoskeleton re-arrangement and prevents the immunological synapse (IS) from forming. The IS needs to be formed to for antigen presenting cells to activate T cells against the presented antigen, i.e. unwanted matter. By preventing soluble CLEVER-1 from binding to RACK1 on activated T cells, we can prevent soluble CLEVER-1 from blocking the formation of the IS, and thus de-activating T cells.

Hence, it is also an object of the present invention to provide a method of controlling immune activation in an individual, wherein binding of soluble CLEVER-1 to Receptor of Activated Protein C Kinase 1 (RACK1) on lymphocytes is prevented.

In order to achieve among others the objects presented above, the invention is characterized by what is presented in the enclosed independent claims. Some preferred embodiments of the invention will be described in the other claims.

According to the present invention, we can maintain T cell activity and immunological homeostasis by blocking soluble CLEVER-1 from binding to RACK1 on lymphocytes. It is also possible to maintain T cell activity and immunological homeostasis by inhibiting and/or neutralizing soluble CLEVER-1 in circulation and/or lymph. In turn, by administering a soluble form of CLEVER-1 in an individual, we can de-activate already activated T cells by binding to RACK1 on lymphocytes and suppress the immune system. Hence, the present invention provides a method for controlling immune activation in an individual, wherein a level of soluble CLEVER-1 in a blood circulation and/or lymph is controlled by affecting the amount of soluble CLEVER-1 in a blood circulation and/or lymph by blocking soluble CLEVER-1 from binding to RACK1 on lymphocytes and/or by administering a soluble form of CLEVER-1. Immune system can be de-activated by increasing a level of soluble CLEVER-1 in blood circulation and/or lymph by administering CLEVER-1 or fragment thereof in a soluble form in an individual. Hence, in an embodiment according to the present invention, CLEVER-1 or fragment thereof in a soluble form can be used as an immunosuppressive agent. Alternatively, a state of immune action can be maintained or gained by neutralizing and/or inhibiting soluble CLEVER-1 in a circulation and/or lymph in an individual by administering an agent capable of inhibiting and/or neutralizing soluble CLEVER-1. According to an embodiment of the present invention, a state of immune action can be controlled, maintained or gained by blocking soluble CLEVER-1 from binding to RACK-1 on lymphocytes by administering an agent capable of binding to RACK-1.

According to a first aspect, the present invention relates to use of CLEVER-1 or fragment thereof in a soluble form, or use of an agent capable of binding to RACK1 on lymphocytes, in controlling immune response in an individual.

According to a second aspect, the present invention relates to use of CLEVER-1 or fragment thereof in a soluble form as an immunosuppressive agent.

According to a third aspect, the present invention relates to a method for controlling immune activation in an individual, wherein a level of soluble CLEVER-1 in a blood circulation and/or lymph is controlled and/or altered by affecting in an amount of soluble CLEVER-1 in a blood circulation and/or lymph. Hence, the present invention provides means to balance immunological homeostasis towards immunosuppression or immune activation by either administering or inducing a soluble form of CLEVER-1 or fragment thereof that will bind to RACK1 on activated lymphocytes and cause inactivation of the immune system, i.e. immunosuppression; or by decreasing levels of soluble CLEVER-1 by administering agent capable of inhibiting and/or neutralizing soluble CLEVER-1 in circulation, and/or by blocking soluble CLEVER-1 from binding to RACK1 on activated lymphocytes to maintain immune activation and the formation of the immunological synapse between lymphocytes and antigen presenting cells.

A method for controlling immune activation or affecting immune response in an individual according to an embodiment of the present invention, wherein the method comprises controlling and/or altering a level of soluble CLEVER-1 in a blood circulation and/or lymph by
administering CLEVER-1 or fragment thereof in a soluble form to an individual, or
administering an agent capable of inhibiting and/or neutralizing soluble CLEVER-1 in an individual, wherein a function of soluble CLEVER-1 is inhibited, and it cannot be bound to a RACK-1 receptor on activated T cells, and/or
administering an agent capable of binding to Receptor of Activated Protein C Kinase 1 (RACK1) on lymphocytes, wherein binding of soluble CLEVER-1 to a RACK-1 receptor is prevented.

According to an embodiment of the present invention, sCLEVER-1 level and/or T cell activation can be analysed from a blood sample obtained from the patient to check need of either immune activation or immunosuppression of T cells in an individual prior to treatment or during treatment period. T cell activation can be analyzed e.g. by increase of biomarker CD69, CXCR3 and/or CD25. A level of sCLEVER-1 can be analyzed e.g. by labeled anti-CLEVER-1 antibody.

A method for controlling immune activation according to the present invention can be used in treatment of various diseases and disorders. As it has been now found that soluble CLEVER-1 levels are increased in cancer patients, T cell activation according to the present invention by an agent capable of binding to Receptor of Activated Protein C Kinase 1 (RACK1) on lymphocytes can be used in the treatment of cancer for maintaining or gaining immune response, but these agents can also be similarly used in T cell activation in the treatment of different kind of infections, inflammations or states of immune exhaustion.

Use of CLEVER-1 or fragment thereof in a soluble form as an immune-suppressive agent can be used in the treatment of different kind of inflammations and autoimmune diseases.

According to the present invention, CLEVER-1 or fragment thereof in a soluble form, or an agent capable of binding to Receptor of Activated Protein C Kinase 1 (RACK1) on lymphocytes, can be used use in therapy by controlling immune response in an individual, wherein a level of soluble CLEVER-1 in a blood circulation and/or lymph is controlled and/or altered.

### DESCRIPTION OF THE DRAWINGS

FIG. 1 shows that sCLEVER-1 levels are increased in cancer patient blood. CLEVER-1 in soluble form (sCLEVER-1) in plasma of normal healthy donors (n=44), breast cancer patients (BC, treatment naive; n=33) and MATINS cancer patients (n=46) measured by Time-resolved fluorescence immunoassay (TRFIA) developed in-house. One-way ANOVA with Dunnett's multiple comparison test. Each dot represents one patient.

FIG. 2 shows forms of soluble CLEVER-1 detected by mean fluorescent intensity (MFI) using a labeled antibody detecting CLEVER-1. AB=apoptopic bodies and cell debris, MVs=micro vesicles, EX=exosomes, FF=free form.

FIG. 3 shows that CLEVER-1 binds RACK1 on CD8+ T-cells and inhibits their proliferation. A: Immunoprecipitation of RACK1 from CD8+ T-cell lysate using placenta purified CLEVER-1 (pClever-1=soluble Clever-1) as bait. B: Dot blot assay showing specific binding of RACK1 on immobilized pClever-1. C: Carboxyfluorescein succinimidyl ester (CFSE) labelled human CD8+ T-cells activated with anti-CD3/CD28 dynabeads and incubated with different concentrations of placenta purified CLEVER-1 or heat inactivated pClever-1 (h-Clever-1). The proliferation was quantified from G4-G6 generations by CFSE dilution. One-way ANOVA, *P<0.05.

FIG. 4 shows that sCLEVER-1 in plasma of MATINS patients at predose (C1D1 (cycle 1, day 1)) and during three weeks on treatment. The drop in sCLEVER-1 levels 24 h after treatment (C1D2 (cycle 1, day 2)) represents anti-CLEVER-1 antibody FP-1305 bound to sCLEVER-1. Each dot represents one patient (n=35).

FIG. 5 shows that RACK1 shows transient cell surface translocation upon CD8+ T-cell activation. A: Confocal images of an activated CD8+ T-cell stained with RACK1 and nuclear stain. B: Cell surface expression of RACK1 analyzed with flow cytometry at indicated time-points after T-cell activation.

### DETAILED DESCRIPTION OF THE INVENTION

The term “soluble CLEVER-1” or “sClever-1” in the present disclosure refers to CLEVER-1 protein present in a soluble form in circulation and/or lymph. Soluble CLEVER-1 is an extracellular CLEVER-1 protein present in a blood circulation and lymph. In circulation soluble CLEVER-1 can be found in exosomes, micro vesicles, free form, and bound to apoptotic bodies and cell debris. Now, it has been found that soluble CLEVER-1 can be used as a target for affecting immune response in an individual.

According to an embodiment of the present invention, CLEVER-1 or its fragment in soluble form that can bind to Receptor of Activated Protein C Kinase 1 (RACK1) on activated lymphocytes can be administered to an individual for controlling immune activation or achieving immunosuppression. In an embodiment of the present invention, soluble CLEVER-1 or its fragment to be administered to an individual has been bound to a carrier. In an embodiment of the present invention CLEVER-1 or its fragment can be bound to liposome or macromolecule in order to provide feasible administration. In carrier structures CLEVER-1 or its fragment maintains its functionality as an immunosuppressive agent. Hence, CLEVER-1 or fragment thereof in a soluble form can be used as an immunosuppressive agent.

According to an aspect of the present invention, the invention relates to a method for blocking soluble CLEVER-1 in circulation from binding to RACK-1 on lymphocytes to gain or maintain a state of immune activation. Soluble CLEVER-1 can be prevented to bind to RACK1 on activated T cells by an agent capable of binding to RACK1 on lymphocytes. According to an embodiment of the present invention an agent capable of binding to RACK1 on lymphocytes can be selected from the group consisting of anti-RACK-1 antibody, a peptide, a small molecule inhibitor, or a fusion protein. An agent capable of binding to RACK1 on lymphocytes can be any suitable agent which can be bound to RACK1 on lymphocytes and thus to prevent binding of soluble CLEVER-1 present in circulation to RACK1 on lymphocytes. According to an aspect of the present invention, the invention relates to an agent capable of binding to RACK1 on lymphocytes for use in the treatment of disease or disorder by achieving immune action of T cells in an individual.

A method of affecting immune response in an individual according to present invention comprises
altering a level of soluble CLEVER-1 in circulation and/or lymph by administering CLEVER-1 or fragment thereof in a soluble form, or administering an agent capable of inhibiting and/or neutralizing soluble CLEVER-1 in a circulation and/or lymph in an individual, or
preventing binding of soluble CLEVER-1 to Receptor of Activated Protein C Kinase 1 (RACK1) on lymphocytes by administering an agent capable of binding to Receptor of Activated Protein C Kinase 1 (RACK1) on lymphocytes,

for achieving immune activation or immunosuppression of T cells in an individual. A method of affecting immune response according to present invention can be used in treatment of various diseases and disorders, wherein immune activation or immunosuppression of T cells is required. Immune activation of T cells by decreasing sClever amount in circulation might be valuable in patients having observed increased amount of sClever-1. A level of soluble CLEVER-1 in circulation and/or lymph can also be altered by administering an agent capable of inhibiting and/or neutralizing soluble CLEVER-1 in a circulation and/or lymph in an individual.

An agent capable of inhibiting and/or neutralizing soluble CLEVER-1 can be used as an immune activating agent in the treatment of various diseases and disorders, wherein immune activation of T cells is required, such as infections, inflammation, cancer or states of immune exhaustion. Neutralizing or inhibiting soluble CLEVER-1 from circulation and/or lymph gain or maintain a state of immune activation. An agent capable of inhibiting and/or neutralizing soluble CLEVER-1 may be selected from the group consisting of an antibody, a peptide, a small molecule inhibitor or a fusion protein. An agent capable of inhibiting and/or neutralizing soluble CLEVER-1 can be any suitable agent which can be bound to soluble CLEVER-1 in circulation and/or lymph and thus to prevent its binding to RACK1 on lymphocytes. An agent capable of inhibiting and/or neutralizing soluble CLEVER-1 can be antiCLEVER-1 antibody. An agent capable of inhibiting and/or neutralizing soluble CLEVER-1 can be a humanized monoclonal anti-CLEVER-1 antibody. Anti-Clever-1 antibody may be a humanized monoclonal immunoglobulin G4K antibody bexmarilimab (International Nonproprietary Name (INN)) as disclosed in WHO Drug Information, Vol. 33, No. 4, 2019), or bexmarilimab variant or the antibody in a bexmarilimab biosimilar. In an embodiment, a bexmarilimab biosimilar comprises a bexmarilimab variant as the drug substance. In an embodiment, a bexmarilimab biosimilar has substantially the same amino add sequence of heavy and light chains as bexmarilimab. As used herein, a “bexmarilimab variant” means an antibody which comprises sequences of heavy chain and light chain that are identical to those in bexmarilimab, except for having one or more conservative amino acid substitutions at positions that are located outside of the light chain CDRs and/or one or more conservative amino acid substitutions that are located outside of the heavy chain CDRs, e.g. the variant positions are located in the framework regions or the constant region. A bexmarilimab variant is substantially the same as bexmarilimab with respect to binding affinity to CLEVER-1.

A cell line producing the therapeutic anti-Clever-1 antibody bexmarilimab (FP-1305) has been deposited on 27 May 2020 under the terms of the Budapest Treaty on the International Recognition of the Deposit of Microorganisms for the Purposes of Patent Procedure with the DSMZ-German Collection of Microorganisms and Cell Cultures GmbH, Inhoffenstrasse 7B, D-38124 Braunschweig, Germany, and has the accession number DSM ACC3361. The present invention is not to be limited in scope by the culture deposited, since the deposited embodiment is intended as a single illustration of one aspect of the invention. The deposit of material herein does not constitute an admission that the written description herein contained is inadequate to enable the practice of any aspect of the invention, including the best mode thereof, nor is it to be construed as limiting the scope of the claims to the specific illustration that it represents.

An agent capable of inhibiting and/or neutralizing soluble CLEVER-1 can also be a peptide, a small molecule inhibitor or a fusion protein.

### Experiments

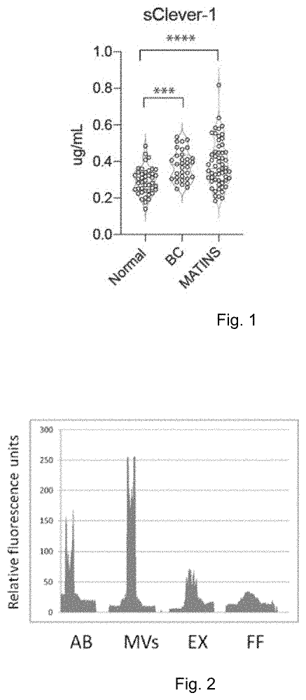

As is shown in FIG. 1, it has been found that CLEVER-1 is present in large quantities in soluble form in the serum and lymph of end-stage cancer patients compared to healthy donors. The results are form a phase I/II first-in-man clinical trial (clinicaltrials.gov NCT03733990: A Study to Evaluate Safety, Tolerability and Preliminary Efficacy of FP-1305 in Cancer Patients (MATINS)), which was initiated to study the safety, tolerability and early efficacy of FP-1305, a humanized anti-Clever-1 antibody, also known as bexmarilimab (International Nonproprietary Name (INN)) as disclosed in WHO Drug Information, Vol. 33, No. 4, 2019).

To evaluate the cellular source of sClever-1, we have purified different fractions of conditioned medium collected from primary cell cultures of lymphatic endothelial cells (LECs) obtained from commercial sources and buffy coat (Finnish Red Cross) derived macrophages using at least four individual donors. The protocol comprises centrifugation to separate debris and apoptotic bodies (2000 G for 10 min, Sorvall RT6000B), micro vesicles (12 900 rpm 30 min, SS-34, Sorvall RC5C and 20 000 G 30 min, Eppendorf centrifuge 5417R), and ultracentrifugation to separate exosomes (32 000 rpm 70 min, Beckman coulter, Optima L-90K). As shown in FIG. 2, most of sClever-1 is found in micro vesicles.

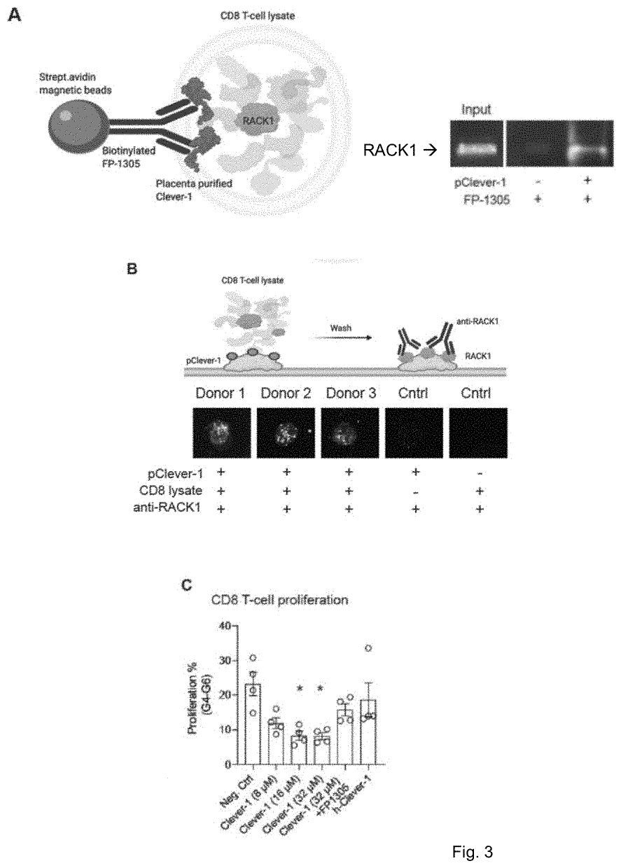

To investigate the binding partner of sClever-1 on lymphocytes, we have utilized CLEVER-1 extracted from human placenta (in-house) in immunoprecipitation assays as a bait to pull-down the possible ligand of CLEVER-1 on CD8+ T-cells. We have analyzed the precipitates with mass spectrometry and found RACK1 (Receptor of activated protein C kinase 1), which is an important component of the immunological synapse between antigen presenting cells and T-cells, as a possible ligand for CLEVER-1. We have validated this interaction by immunoprecipitation and dot blot assays (FIGS. 3A and 3B). As shown in FIGS. 3A and 3B, RACK1 bound to placenta purified CLEVER-1, confirmed by anti-RACK1. Hence, it can be concluded that CLEVER-1 binds to activated T cells using Receptor of Activated Protein C Kinase 1 (RACK1) as its ligand. Functionally the placenta purified CLEVER-1 (i.e. sClever-1) was able to inhibit T-cell proliferation as shown in FIG. 3C, which was reversed by neutralizing its effect with antiCLEVER-1 antibody FP-1305. Hence, it can be concluded that extracellular CLEVER-1 interferes in the formation of the immunological synapse by binding to RACK-1 on CD8+ T-cells.

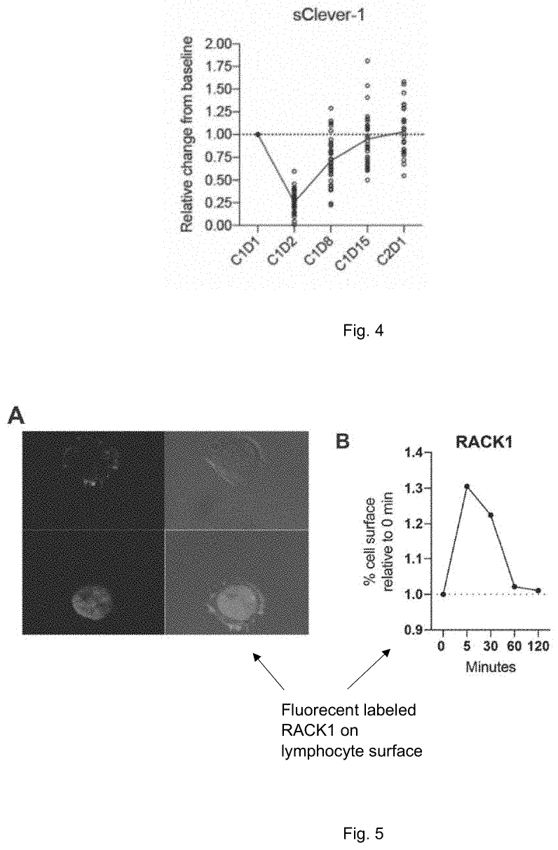

FIG. 4 presents that administration of anti-CLEVER-1 antibody FP-1305 to the patients in MATINS trial led to a dramatic decrease in the free form of sClever-1 24h after treatment (C1D2) indicating that a major part of FP-1305 was binding to soluble Clever-1 species. During treatment after the administration of FP-1305 and without any further administrations, the sClever-1 levels are slightly increased. In FIG. 4, C refers to treatment cycle and D refers to day.

RACK1 is an intracellular molecule and FIG. 5 shows that in activated CD8⁺ T cells RACK1 is brought to the lymphocyte surface during its activation and hence sClever-1 can directly bind to it. This phenomenon has confirmed by activating T-cells with dynabeads and measured cell surface RACK1 levels thereafter with flow cytometry. As shown in FIG. 5A, RACK1 shows transient cell surface translocation upon CD8⁺ T-cell activation.

Hence, on the basis of the results presented above it can be concluded that sClever-1 is present as increased amounts in circulation in cancer patients, but also other states having immune exhaustion. It is now found that sClever-1 can be bound to activated T cells using Receptor of Activated Protein C Kinase 1 (RACK1) as its ligand and hence it can affect T cell activation by preventing the immunological synapse (IS) from forming. When inhibiting and/or neutralizing sClever-1 or blocking its binding to RACK1, T cells activation can be maintained and/or controlled. It has also been found and confirmed that sClever-1 has an ability to inhibit T-cell proliferation (FIG. 3C) and so functioning as an immunosuppressive agent.

1. A method for controlling immune activation or affecting immune response in an individual, wherein the method comprises controlling and/or altering a level of soluble CLEVER-1 in a blood circulation and/or lymph
by administering CLEVER-1 or fragment thereof in a soluble form, or
by administering an agent capable of binding to Receptor of Activated Protein C Kinase 1 (RACK1) on lymphocytes, and/or
by administering an agent capable of inhibiting and/or neutralizing soluble CLEVER-1 in a circulation and/or lymph in an individual.

2. The method according to claim 1, wherein the agent capable of binding to Receptor of Activated Protein C Kinase 1 (RACK1) on lymphocytes is selected from the group consisting of an antibody, a peptide, a small molecule inhibitor or a fusion protein.

3. The method according to claim 1, wherein the agent capable of inhibiting and/or neutralizing soluble CLEVER-1 is selected from the group consisting of an antibody, a peptide, a small molecule inhibitor or a fusion protein.

4. The method according to claim 3, wherein the agent capable of inhibiting and/or neutralizing soluble CLEVER-1 comprises a humanized monoclonal immunoglobulin G4K antibody bexmarilimab.

5. CLEVER-1 or fragment thereof in a soluble form for use as an immunosuppressive agent.

6. CLEVER-1 or fragment thereof in a soluble form according to claim 5 for use as an immunosuppressive agent, wherein CLEVER-1 or fragment thereof has been bound to a carrier.
